(19) United States
(12) Patent Application Publication
(10) Pub. No.: US 2024/0296154 A1
(43) Pub. Date: Sep. 5, 2024

(54) TREE BASED DETECTION OF DIFFERENCES IN DATA

(71) Applicant: Express Scripts Strategic Development, Inc., St. Louis, MO (US)

(72) Inventor: Nilay Mukund Sundarkar, Georgetown, TX (US)

(21) Appl. No.: 18/117,054

(22) Filed: Mar. 3, 2023

(51) Int. Cl. G06F 16/23; G06F 16/22; G06F 16/27; G16H 10/60

(52) U.S. Cl. CPC G06F 16/2365; G06F 16/2246; G06F 16/2255; G06F 16/27; G16H 10/60

(57) ABSTRACT

Methods and systems are provided for performing operations comprising: accessing, by one or more processors, a first set of data from a first source and a second set of data from a second source; extracting, based on a key, a first subset of data from the first set of data and a second subset of data from the second set of data; generating a first Merkle tree based on the first subset of data and a second Merkle tree based on the second subset of data; comparing a first node from the first Merkle tree with a corresponding second node from the second Merkle tree; and identifying one or more differences between the first subset of data and the second subset of data in response to determining that the first node fails to match the second node.

### BACKGROUND

Users are increasingly using the Internet, such as websites, to access information and perform transactions. As more and more services become available over the Internet, data generated by such services continues to grow and be stored in a wide range of storage locations. Sometimes the data is replicated across multiple servers and ensuring the replicated data is accurate is has become an area of concern.

### BRIEF DESCRIPTION OF THE DRAWINGS

FIG. 1 is a block diagram of an example difference identification system, according to some embodiments.

FIG. 2 is an example database that may be deployed within the system of FIG. 1, according to some embodiments.

FIG. 3 illustrates example implementation of the difference identification system of FIG. 1, according to some embodiments.

FIG. 4 illustrates an example Merkle tree generated by the system of FIG. 1, according to some embodiments.

FIGS. 5A and 5B are example traversals of Merkle trees to perform difference identification by the system of FIG. 1, according to example embodiments.

FIG. 6 is a flowchart illustrating example operations of the difference identification system, according to example embodiments.

FIG. 7 is a block diagram illustrating an example software architecture, which may be used in conjunction with various hardware architectures herein described.

FIG. 8 is a block diagram illustrating components of a machine, according to some example embodiments.

### DETAILED DESCRIPTION

Example methods and systems for a difference identification system are described. In the following description, for purposes of explanation, numerous specific details are set forth in order to provide a thorough understanding of example embodiments. It will be evident, however, to one of ordinary skill in the art that embodiments of the invention may be practiced without these specific details.

Online transactions typically consume resources of one or more servers. Such resources include memory allocated to various services hosted by the servers. The amount of memory that each server can allocate is typically physically limited. As such, over time, the servers need to redistribute the data across other servers. This can also be performed to increase data throughput by allowing a client device to access data from other sources. Ensuring that the data that is replicated across the servers is identical and lacks differences is usually incredibly complex, time consuming, and consumes a great deal of resources. Typical systems actively and routinely check differences between the data replicated or provided by different sources in a multi-step process. Namely, the typical systems generate a key and obtain a set of data corresponding to the key from each of the data sources. The obtained data from each of the sources is then compared on an individual portion-by-portion basis which can take up to a number of comparisons equal to the quantity of portions. As data portions increase in size, the number of comparisons also increase by the same proportion. Also, even if there do not exist any difference in the data, the typical systems still need to compare each individual data portion to make the determination that no differences exist. This makes such systems incredibly inefficient and impractical to use for large data files and comparisons.

The disclosed embodiments provide systems and methods to identify differences in data from different sources in a faster and more efficient manner. Particularly, the disclosed embodiments leverage multiple Merkle trees to represent the data from a variety of sources, including database sources and/or real-time sources, such as topic sources. For example, a first Merkle tree can be generated to represent data from a first data source and a second Merkle tree can be generated to represent data from a second data source. The nodes of the first and second Merkle trees can then be compared to determine whether differences in data sources exist at all and to then locate the specific portions that differ in the data.

By using the Merkle trees to detect existence of differences in data across data sources, the disclosed embodiments detect existence of differences with a single comparison operation of root nodes of the Merkle trees which decreases the complexities encountered by typical systems logarithmically and provides results at least an order of magnitude faster than typical systems. Once the existence of a difference in the data sources is detected, finding the source of the difference or the specific data portion that is different between the two data sources can also be performed in a fast and efficient manner without having to compare each and every one of the data portions. Particularly, nodes of the first and second Merkle trees that are not different can be ignored in further comparisons and only nodes of the first and second Merkle trees that differ from each other need to be considered. A Merkle tree is a tree in which every “leaf” (node) is labelled with the cryptographic hash of a data block, and every node that is not a leaf (called a branch, inner node, or inode) is labelled with the cryptographic hash of the labels of its child nodes.

This reduces the overall number of resources needed to detect differences in data sets and allows the disclosed embodiments to identify sources of the differences more quickly and efficiently that typical systems. Also, because the differences can be detected so quickly, the disclosed embodiments can operate on real-time data received from different sources to detect differences in real-time data which is impractical to perform with typical systems.

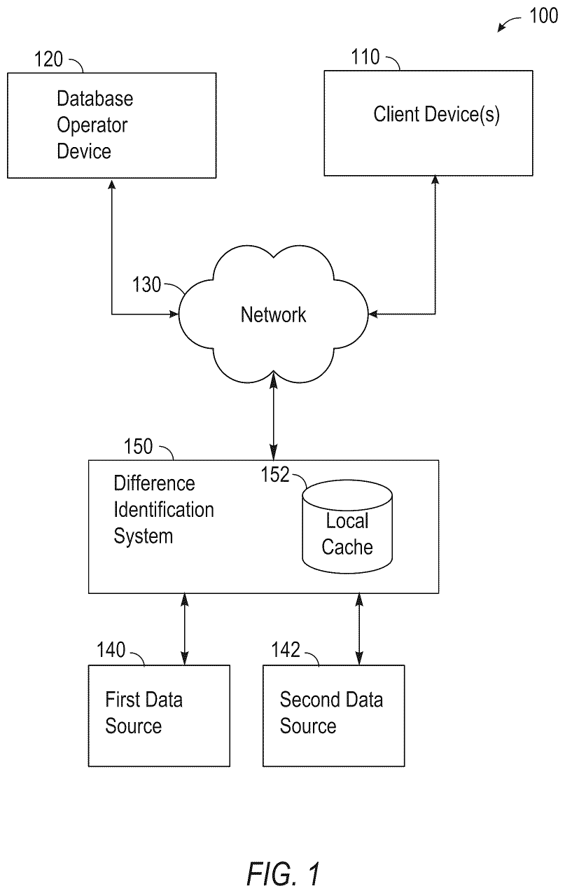

FIG. 1 is a block diagram showing an example system 100 according to various exemplary embodiments. The system 100 includes one or more client devices 110, a database operator device 120, a difference identification system 150, a first data source 140, and a second data source 142 that are communicatively coupled over a network 130 (e.g., Internet, telephony network).

As used herein, the term “client device” may refer to any machine that interfaces to a communications network (such as network 130) to obtain data from one or more first data source 140 or second data source 142. The client device 110 may be, but is not limited to, a mobile phone, desktop computer, laptop, portable digital assistants (PDAs), smart phones, a wearable device (e.g., a smart watch), tablets, ultrabooks, netbooks, laptops, multi-processor systems, microprocessor-based or programmable consumer electronics, game consoles, set-top boxes, or any other communication device that a user may use to access a network or a service hosted by the servers, such as first data source 140 or second data source 142.

The network 130 may include, or operate in conjunction with, an ad hoc network, an intranet, an extranet, a virtual private network (VPN), a local area network (LAN), a wireless network, a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other type of cellular or wireless coupling. In this example, the coupling may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, fifth generation wireless (5G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

The first data source 140 can be a first database or real-time data source, such as a topic source. The first data source 140 can host various information or provide various information, such as patient information including health claim information or physician or hospital information. In certain cases, the first data source 140 may receive a request from the difference identification system 150 to replicate data to another data source, such as the second data source 142. The second data source 142 can be a second database or real-time data source, such as a topic source. The second data source 142 can be hosted on the same or different server as the first data source 140.

In the process of replicating data from the first data source 140 to the second data source 142, the difference identification system 150 may verify that errors have not been introduced. Particularly, the difference identification system 150 ensures that the data that is present on both the first data source 140 and the second data source 142 is identical. In some instances, in case of transformations applied during data replication, a similar transformation can be applied to either the source or the target data before extracting the values into a Merkel tree. In certain cases, the difference identification system 150 performs such verifications on a real-time basis, periodically, and/or in response to detecting events in data that is stored and/or provided by the first data source 140 and/or the second data source 142. In such cases, the difference identification system 150 accesses or updates Merkle trees that represent the data from the first data source 140 and the second data source 142 and then traverses each of the Merkle trees to detect existence of any errors or differences and the specific data portions that cause the errors or differences.

Specifically, the difference identification system 150 accesses a first set of data from a first source (e.g., the first data source 140) and a second set of data from a second source (e.g., the second data source 142). The difference identification system 150 extracts, based on a key, a first subset of data from the first set of data and a second subset of data from the second set of data and generates a first Merkle tree based on the first subset of data and a second Merkle tree based on the second subset of data. The difference identification system 150 compares a first node (e.g., a root node) from the first Merkle tree with a corresponding second node (e.g., a root node) from the second Merkle tree. The two trees can be traversed in a breadth first manner. The difference identification system 150 identifies one or more differences between the first subset of data and the second subset of data in response to determining that the first node fails to match the second node.

Merkle tree generation for the source and target values is done by making sure the values in the source and target trees are for the same configuration map (keys) and the leaf nodes are stored in a sorted manner. This ensures that the generated hashes in the intermediate root nodes and the root node do not differ due to inconsistent order of generating the hashes between the source and the target Merkle trees.

In case the JSON has an array node, the library also provides a way to specify logical keys to individual array element so that if the order of such array elements is not consistent between the source and the target JSONs, the library can then sort the values extracted from such array elements by the logical key to the array. This ensures that the generated hashes in the intermediate root nodes and the root node do not differ due to inconsistent order of generating the hashes between the source and the target Merkle trees.

In some examples, the first source includes at least one of a first database or a first streaming source including a first topic and the second source includes at least one of a second database or a second streaming source including a second topic. In some examples, the first set of data includes a first set of JSON files and the second set of data includes a second set of JSON files.

In some examples, the difference identification system 150 receives the key, such as from the database operator device 120 and/or the one or more client devices 110 or other source that identifies and detects an event associated with the data stored by the first data source 140 and/or the second data source 142. The difference identification system 150 accesses a configuration map that is associated with the key and extracts the first subset of data and the second subset of data according to the configuration map. In some examples, the configuration map includes a plurality of keys that define an organization of the extracted first subset of data and the extracted second subset of data.

In some examples, the difference identification system 150 generates the first Merkle tree by generating a first hash value based on a first portion of the first subset of data. The difference identification system 150 generates a second hash value based on a second portion of the first subset of data. The difference identification system 150 generates a first root node of the first Merkle tree including a third hash value representing a combination of the first hash value and the second hash value and links the first root node to the first portion of the first subset of data and the second portion of the first subset of data.

In some examples, the difference identification system 150 generates a fourth hash value based on a third portion of the first subset of data and generates a fifth hash value based on a fourth portion of the first subset of data. The difference identification system 150 generates a second root node of the first Merkle tree including a sixth hash value representing a combination of the fourth hash value and the fifth hash value and links the second root node to the third portion of the first subset of data and the fourth portion of the first subset of data. In some aspects, the difference identification system 150 generates a third root node of the first Merkle tree including a seventh hash value representing a combination of the third hash value and the sixth hash value; and linking the third root node to the first root node and the second root node.

In some examples, the difference identification system 150 determines existence of the one or more differences between the first subset of data and the second subset of data in response to determining that the first node fails to match the second node. In response to determining the existence of the one or more differences, the difference identification system 150 identifies the one or more differences between the first subset of data and the second subset of data by comparing additional nodes of the first Merkle tree with additional nodes of the second Merkle tree. In some aspects, the difference identification system 150 compares the additional nodes of the first Merkle tree with the additional nodes of the second Merkle tree by traversing a first subset of nodes of the first Merkle tree without traversing a second subset of nodes of the first Merkle tree and traversing a third subset of nodes of the second Merkle tree without traversing a fourth subset of nodes of the second Merkle tree.

In some examples, the difference identification system 150 determines that at least one node of the first subset of nodes is different from at least one node of the third subset of nodes. The difference identification system 150 determines that a root node the second subset of nodes is identical to a root node of the fourth subset of nodes and, in response to determining that the root node the second subset of nodes is identical to the root node of the fourth subset of nodes, selects paths along the first and second Merkle trees to traverse that include the first and third subsets of nodes.

In some examples, the difference identification system 150 identifies a first plurality of nodes of the first Merkle tree that branch off the first node along a first path and identifies a second plurality of nodes of the first Merkle tree that branch off the first node along a second path. The difference identification system 150 identifies a third plurality of nodes of the second Merkle tree that branch off the second node along a third path and identifies a fourth plurality of nodes of the second Merkle tree that branch off the second node along a fourth path.

In some examples, the difference identification system 150 compares a first hash value stored in a third node of the first plurality of nodes with a second hash value stored in a fourth node of the third plurality of nodes. The difference identification system 150 compares a third hash value stored in a fifth node of the second plurality of nodes with a fourth hash value stored in a sixth node of the fourth plurality of nodes and determines that the first hash value matches the second hash value and that the third hash value fails to match the fourth hash value. The difference identification system 150, in response to determining that the first hash value matches the second hash value and that the third hash value fails to match the fourth hash value, processes the second plurality of nodes along the second path and the fourth plurality of nodes along the fourth path. The difference identification system 150 discontinues or prevents processing the first plurality of nodes along the first path and the third plurality of nodes along the third path in response to determining that the first hash value matches the second hash value and that the third hash value fails to match the fourth hash value.

In some examples, the difference identification system 150 identifies a first pair of leaf nodes that are linked to an individual node of the second plurality of nodes that stores a different hash value from an individual node of the fourth plurality of nodes, the individual node of the fourth plurality of nodes being linked to a second pair of leaf nodes. The difference identification system 150 compares data values associated with the first pair of leaf nodes with data values associated with the second pair of leaf nodes to identifying the one or more differences between the first subset of data and the second subset of data.

In some aspects, the first set of data includes a first set of patient profile information and the second set of data includes a second set of patient profile information. In some aspects, the first set of data can be replicated from the first source to the second source which generates the second set of data. In such cases, the difference identification system 150 identifies the one or more differences between the first subset of data and the second subset of data.

In some examples, the difference identification system 150 receives a plurality of events corresponding to different keys. The difference identification system 150 extracts different subsets of data from the first set of data for each of the plurality of events based on a respective one of the keys and extracts different subsets of data from the second set of data for each of the plurality of events based on the respective one of the keys. The difference identification system 150 generates a first plurality of Merkle trees based on the different subsets of data from the first set of data for each of the plurality of events and generates a second plurality of Merkle trees based on the different subsets of data from the second set of data for each of the plurality of events. In some examples, the difference identification system 150 detects differences between the different subsets of data of the first and second sets of data by comparing the first plurality of Merkle trees with the second plurality of Merkle trees.

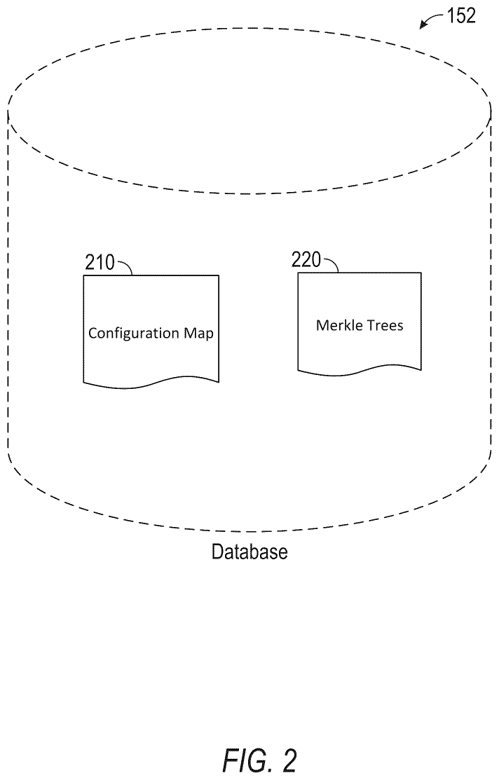

FIG. 2 is an example local cache 152 that may be deployed within the system of FIG. 1, according to some embodiments. As shown, the local cache 152 includes configuration map 210 and Merkle trees 220. While the configuration map 210 is shown as part of the local cache 152, the configuration map 210 can be stored outside of the database and remain in the application configuration properties. The configuration map 210 can be loaded in the memory at the time of start up and helper classes can be used to generate the configuration based on a schema of a given file, such as a JSON file. The configuration map 210 stores parameters for extracting and organizing data associated with different keys. For example, a first key that is stored in the configuration map 210 can be associated with a first set of data elements that need to be extracted from a data corpus, such as from first data source 140. The first key in the configuration map 210 can indicate how the data that is extracted from the data corpus is to be organized and sorted. By using the data keys stored in the configuration map 210, data that is extracted from two different data sources or corpuses, such as first data source 140 and second data source 142 can be organized and sorted in a similar fashion to simplify and expedite Merkle tree generation and comparison.

The Merkle trees 220 stores a plurality of Merkle trees each associated with a different key that is stored in the configuration map 210 and each generated based on a respective corpus of data, such as data provided by the first data source 140 and data provided by the second data source 142. The difference identification system 150 generates a first Merkle tree by extracting a first set of data from the first data source 140 using a key according to the organization of the data for the key stored in the configuration map 210. The first Merkle tree can be serialized and stored as one of the Merkle trees 220. The difference identification system 150 generates a second Merkle tree by extracting a second set of data from the second data source 142 using the same key according to the organization of the data for the key stored in the configuration map 210. The second Merkle tree can be serialized and stored as one of the Merkle trees 220. Additional keys can be received and used to generate additional Merkle trees 220 by extracting the data from the first data source 140 and the second data source 142.

In some cases, in response to detecting an event, the Merkle trees of the first data source 140 and the second data source 142 associated with a key corresponding to the event can be retrieved from the Merkle trees 220. As explained below, the nodes of the retrieved Merkle trees can be compared starting with the root nodes of the trees. If the root nodes store the same values (e.g., hash values), the difference identification system 150 determines that there is no difference in the data corresponding to the key that is provided by the first data source 140 and the second data source 142. If the root nodes store the different values (e.g., hash values), the difference identification system 150 determines that there exists a difference in the data corresponding to the key that is provided by the first data source 140 and the second data source 142. In such cases, the difference identification system 150 selectively traverses certain subsets of the Merkle trees based on paths corresponding to differences in nodes between the Merkle trees of the different data sources. The Merkle trees continue to be traversed until the specific leaves that represent the data portions that are the sources of the difference are reached and used to actually compare the underlying data to identify the differences in the data. By processing the Merkle trees in this manner, the speed at which differences in data are detected is reduced exponentially at least by an order of magnitude over comparing each individual portion one-by-one as is typically performed and the complexity is reduced substantially. This improves the overall functioning of the device and increases the overall efficiency of the system.

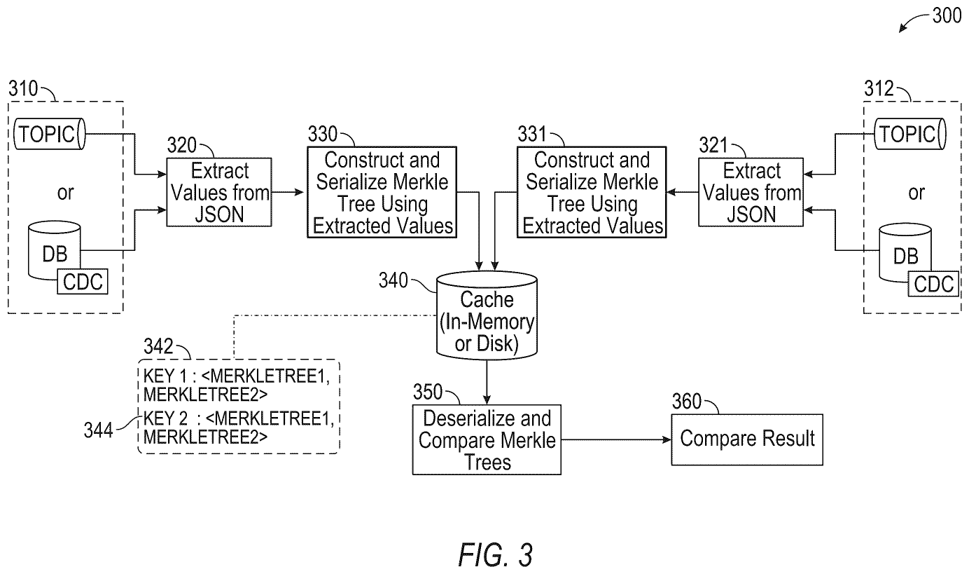

FIG. 3 illustrates example implementation of the difference identification system 300 of the system 100 of FIG. 1, according to some embodiments. The difference identification system 300 includes a first data source 310 which can correspond to the first data source 140 and a second data source 312 which can correspond to the second data source 142. The first data source 310 can include a database and/or a topic (real-time data source or streaming data source) and similarly the second data source 312 can include another database and/or another topic (real-time data source or streaming data source).

The difference identification system 300 can receive an instruction to replicate the database of the first data source 310 into a topic of the second data source 312 and/or to replicate the database of the first data source 310 into the database of the second data source 312 and/or the topic of the first data source 310 into the topic or database of the second data source 312. After replicating the data, the difference identification system 300 can detect an event associated with the data stored or provided by the first data source 310 and/or the second data source 312 that causes the difference identification system 300 to determine whether the data of these sources include differences. To simplify and expedite the process of detecting differences in the data, the difference identification system 300 can generate a plurality of Merkle trees for the data sets in the first data source 310 and the second data source 312.

In some examples, the difference identification system 300 accesses a key that is used to perform a select operation on the data from the first data source 310 and the second data source 312. Using the key, the difference identification system 300 extracts values from the first data source 310, such as from a JSON file that stores the values of the first data source 310. This process can be performed by a data extraction module 320. Simultaneously, before or after this operation, the difference identification system 300 also extracts values from the second data source 312, such as from a JSON file that stores the values of the second data source 312. This process can be performed by a data extraction module 321 which can be the same or a different data extraction module as the data extraction module 320. The data extraction module 321 and the data extraction module 320 can be implemented by the same server and/or by different servers that store the respective data sets of the first data source 310 and the second data source 312.

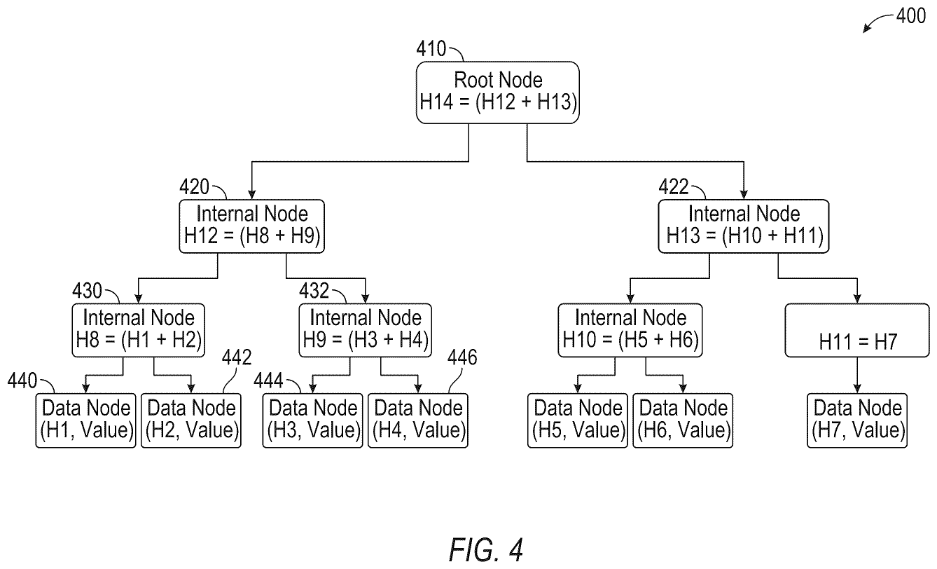

After generating a vector of values from the first data source 310 corresponding to the received key, the difference identification system 300 instructs the Merkle tree module 330 to generate a first Merkle tree using the generated vector. This first Merkle tree is then stored in association with the key (e.g., a key identifier 344) in a database entry 342 of a storage device 340. To generate the first Merkle tree, the Merkle tree module 330 can obtain a set of data elements of the vector and can generate a hash value for each of the data elements. Specifically, as shown in the example Merkle tree 400 of FIG. 4, the Merkle tree module 330 generates a first hash value 440 corresponding to a first data element of the vector of values extracted from the first data source 310 using the received key, a second hash value 442 corresponding to a second data element of the vector of values extracted from the first data source 310 using the received key, a third hash value 446 corresponding to a third data element of the vector of values extracted from the first data source 310 using the received key, and a fourth hash value 446 corresponding to a fourth data element of the vector of values extracted from the first data source 310 using the received key. This process is repeated until all of the data elements of the vector of values is processed to generate the hash values. These hash values form the leaf nodes of the Merkle tree 400.

After generating the leaf nodes of the Merkle tree 400, the Merkle tree module 330 links each adjacent pair of leaf nodes to a respective root node of the Merkle tree 400. For example, the Merkle tree module 330 obtains the first hash value 440 stored in a first leaf node and combines the first hash value 440 with the second hash value 442 that is stored in the adjacent second leaf node. Specifically, the Merkle tree module 330 can add the first hash value 440 with the second hash value 442. The Merkle tree module 330 stores this combined hash value in a first root node 430. Similarly, the Merkle tree module 330 obtains the third hash value 444 stored in a third leaf node and combines the third hash value 444 with the fourth hash value 446 that is stored in the adjacent fourth leaf node. Specifically, the Merkle tree module 330 can add the third hash value 444 with the fourth hash value 446. The Merkle tree module 330 stores this combined hash value in a second root node 432. The Merkle tree module 330 links the first root node 430 with the first and second leaf nodes and links the second root node 432 with the third and fourth leaf nodes of the Merkle tree. The Merkle tree module 330 repeats this process until all of the hash values of the leaf nodes have been combined into a respective root node.

The Merkle tree module 330 then obtains the hash value stored in the first root node 430 and combines the hash value with the hash value that is stored in the adjacent second root node 432. Specifically, the Merkle tree module 330 can add the hash value stored in the first root node 430 with the hash value stored in the second root node 432. The Merkle tree module 330 stores this combined hash value in a third root node 420 and links this third root node 420 with the first root node 430 and second root node 432. This process is similarly performed on other root nodes to generate other root nodes, such as the root node 422. Once only two root nodes remain, such as the root node 420 and the root node 422, the Merkle tree module 330 combines the hash values of those last two root nodes 420 and 422 into the topmost root node 410 of the Merkle tree 400 (e.g., by adding the hash values of the two root nodes 420 and 422) and links the topmost root node 410 with the last two root nodes 420 and 422.

The Merkle tree module 330 performs similar operations to generate the second Merkle tree based on a vector of values from the second data source 312 corresponding to the received key. Each of the first and second Merkle trees is then stored in association with the received key 344. Referring back to FIG. 3, the difference identification system 300 can detect an event that triggers a comparison of the first and second Merkle trees that are stored in association with the received key 344. Specifically, in response to detecting the event, the difference identification system 300 can use the Merkle tree comparison module 350 to access the Merkle trees associated with the first data source 310 and the second data source 312 to detect existence of any differences in the data associated with the received key 344 and to identify the specific data entries that result in such differences, if any.

In some examples, the Merkle tree comparison module 350 identifies a key associated with a detected event. After identifying the key, the Merkle tree comparison module 350 accesses the storage device 340 and searches the database entry 342 to identify the key in the database entry 342. The Merkle tree comparison module 350 then retrieves the Merkle trees stored in association with the key from the database entry 342 and deserializes the Merkle trees. The Merkle tree comparison module 350 traverses the retrieved Merkle trees according to specified paths corresponding to node differences to detect the existence of differences in the underlying data and to identify the specific data entry that cause the differences.

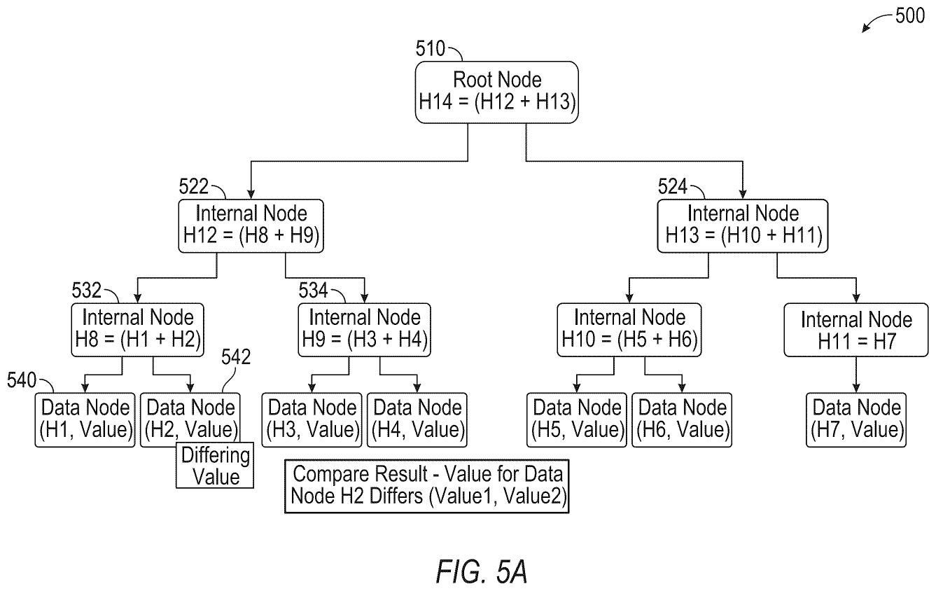

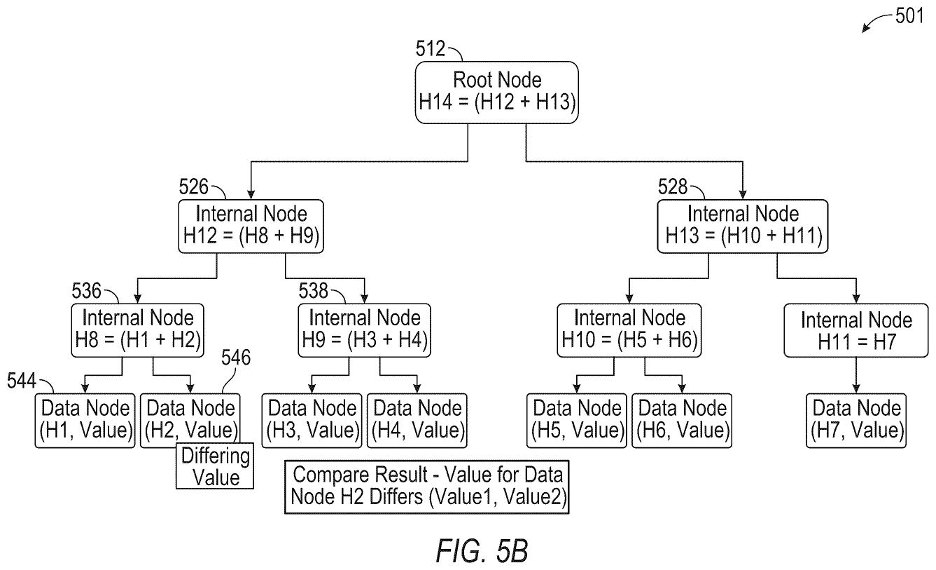

FIGS. 5A and 5B are example traversals of Merkle trees to perform difference identification by the system 100 of FIG. 1, according to example embodiments. For example, the Merkle tree comparison module 350 obtains a first Merkle tree 500 (FIG. 5A) from the storage device 340 and obtains a second Merkle tree 501 (FIG. 5B) from the storage device 340. The first Merkle tree 500 represents data elements of the first data source 310 corresponding to the identified key which have been used to generate the first Merkle tree 500. The second Merkle tree 501 represents data elements of the second data source 312 corresponding to the identified key which have been used to generate the second Merkle tree 501.

In some examples, to detect existence of differences, the Merkle tree comparison module 350 compares the topmost node 510 of the first Merkle tree 500 with the corresponding topmost node 512 of the second Merkle tree 501. In response to determining that the topmost node 510 is identical to the corresponding topmost node 512, the Merkle tree comparison module 350 determines that there do not exist any differences in the data of the first data source 310 and the second data source 312 corresponding to the key (e.g., the data portions are identical). In response to determining that the topmost node 510 is different from the corresponding topmost node 512, the Merkle tree comparison module 350 determines that exists at least one difference in the data of the first data source 310 and the second data source 312 corresponding to the key. In such cases, the Merkle tree comparison module 350 accesses the hash values stored in the next level of the first and second Merkle trees 500 and 501.

For example, the Merkle tree comparison module 350 obtains the hash value stored in the node 522 and the hash value stored in the node 524 which are linked to the topmost node 510 of the first Merkle tree 500. The Merkle tree comparison module 350 obtains the hash value stored in the node 526 and the hash value stored in the node 528 which are linked to the topmost node 512 of the second Merkle tree 501. The Merkle tree comparison module 350 compares the hash value stored in the node 524 with the hash value stored in the node 528. In response to determining that the hash value stored in the node 524 matches or is identical to the hash value stored in the node 528, the Merkle tree comparison module 350 discontinues processing and analyzing nodes that are linked to the nodes 522 and 526, respectively and discontinues traversing further nodes of the Merkle trees 500 and 501 that are linked to the nodes 522 and 526, respectively.

The Merkle tree comparison module 350 obtains the hash value stored in the node 532 and the hash value stored in the node 534 which are linked to the node 522 of the first Merkle tree 500. The Merkle tree comparison module 350 obtains the hash value stored in the node 536 and the hash value stored in the node 538 which are linked to the node 526 of the second Merkle tree 501. The Merkle tree comparison module 350 compares the hash value stored in the node 532 with the hash value stored in the node 536. In response to determining that the hash value stored in the node 532 fails to match or is different from the hash value stored in the node 536, the Merkle tree comparison module 350 continues analyzing and traversing further nodes of the Merkle trees 500 and 501 that are linked to the nodes 532 and 536, respectively. The Merkle tree comparison module 350 compares the hash value stored in the node 534 with the hash value stored in the node 538. In response to determining that the hash value stored in the node 534 matches or is identical to the hash value stored in the node 538, the Merkle tree comparison module 350 discontinues processing and analyzing nodes that are linked to the nodes 526 and 522, respectively and discontinues traversing further nodes of the Merkle trees 500 and 501 that are linked to the nodes 526 and 522, respectively.

The Merkle tree comparison module 350 determines that the nodes 532 and 536 are the last root nodes in the Merkle trees 500 and 501 that were in the path of comparisons to identify the source of the differences in the data from the first data source 310 and second data source 312. In such cases, the Merkle tree comparison module 350 obtains the hash values stored in the leaf nodes linked respectively to the nodes 532 and 536. For example, the Merkle tree comparison module 350 obtains the hash values stored in the leaf nodes 540 and 544 and compares those two hash values. If the two hash values are the same, the Merkle tree comparison module 350 determines that the difference exists in the data entries corresponding to the leaf nodes 542 and 546. In such cases, the Merkle tree comparison module 350 obtains the data entries of the leaf nodes 542 and 546 from the storage device 340 and compares the two data entries to identify the differences in the data and outputs the difference as a comparison result 360. The difference identification system 300 can then update one of data entries corresponding to the difference of the first data source 310 and second data source 312 to match to resolve the error or difference in the data

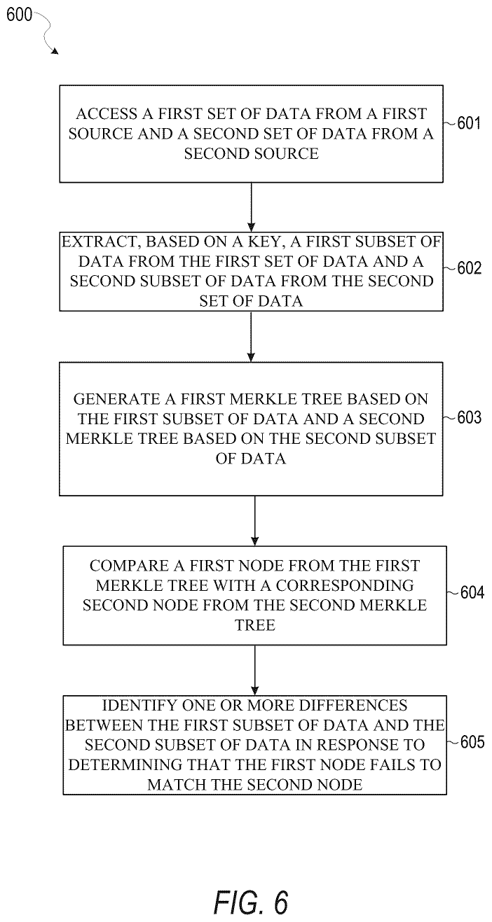

FIG. 6 is a flowchart illustrating example operations of the difference identification system in performing process 600, according to example embodiments. The process 600 may be embodied in computer-readable instructions for execution by one or more processors such that the operations of the process 600 may be performed in part or in whole by the functional components of the system 100; accordingly, the process 600 is described below by way of example with reference thereto. However, in other embodiments, at least some of the operations of the process 600 may be deployed on various other hardware configurations. Some or all of the operations of process 600 can be in parallel, out of order, or entirely omitted.

At operation 601, the difference identification system 150 accesses a first set of data from a first source and a second set of data from a second source, as discussed above.

At operation 602, the difference identification system 150 extracts, based on a key, a first subset of data from the first set of data and a second subset of data from the second set of data, as discussed above.

At operation 603, the difference identification system 150 generates a first Merkle tree based on the first subset of data and a second Merkle tree based on the second subset of data, as discussed above.

At operation 604, the difference identification system 150 compares a first node from the first Merkle tree with a corresponding second node from the second Merkle tree, as discussed above. Specifically, the difference identification system 150 can compare the root node of the first and second Merkle trees to see if all the elements match (e.g., to determine whether the root hash is a match). If not, the difference identification system 150 performs a breadth first traversal of the child nodes, ignoring the nodes (and the nodes below those nodes) that match in their values. This ensures a logarithmic time complexity.

At operation 605, the difference identification system 150 identifies one or more differences between the first subset of data and the second subset of data in response to determining that the first node fails to match the second node, as discussed above.

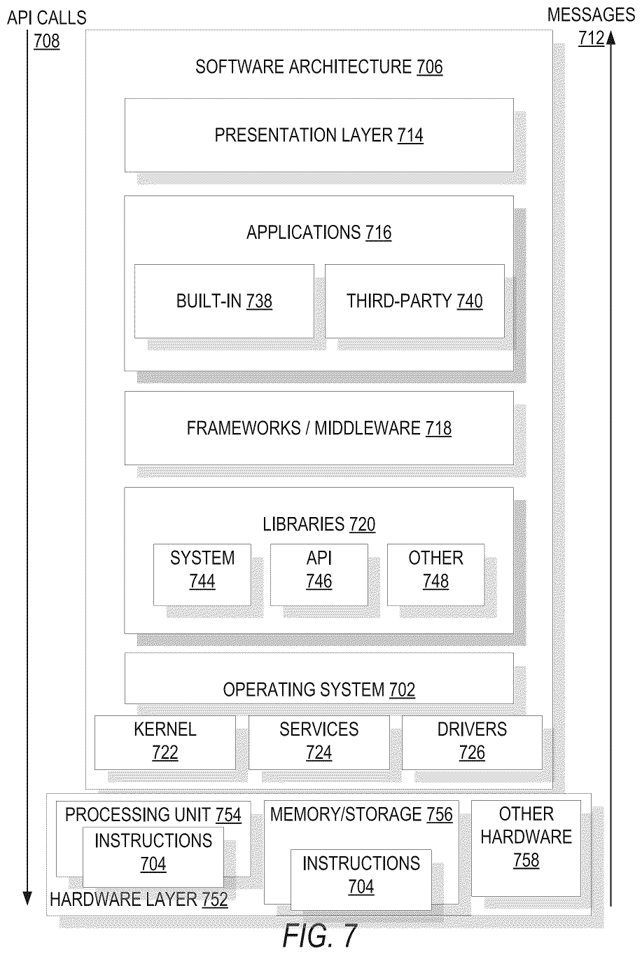

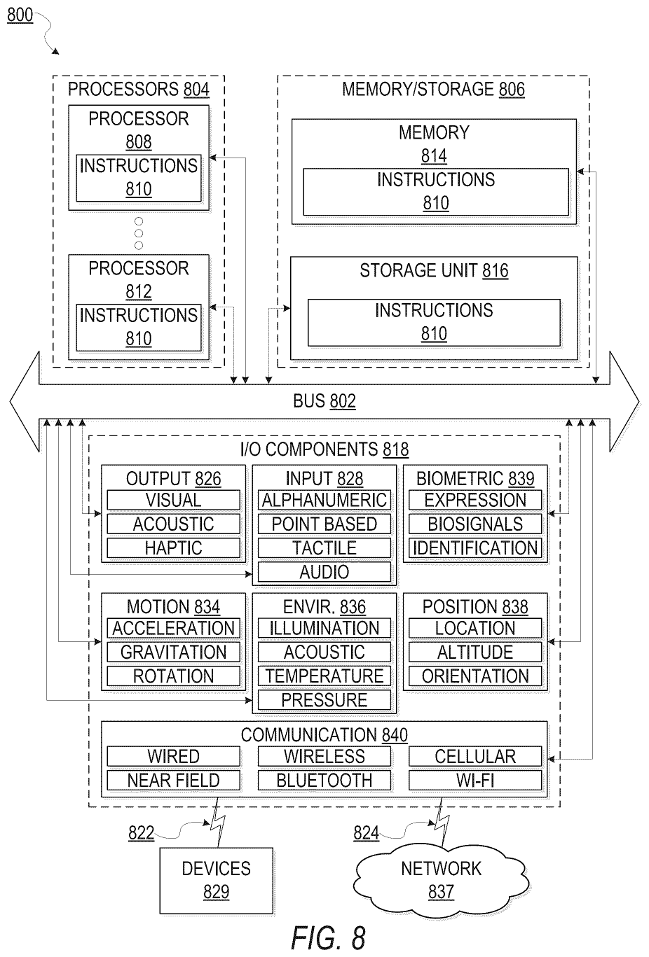

FIG. 7 is a block diagram illustrating an example software architecture 706, which may be used in conjunction with various hardware architectures herein described. FIG. 7 is a non-limiting example of a software architecture and it will be appreciated that many other architectures may be implemented to facilitate the functionality described herein. The software architecture 706 may execute on hardware such as machine 800 of FIG. 8 that includes, among other things, processors 804, memory 814, and input/output (I/O) components 818. A representative hardware layer 752 is illustrated and can represent, for example, the machine 800 of FIG. 8. The representative hardware layer 752 includes a processing unit 754 having associated executable instructions 704. Executable instructions 704 represent the executable instructions of the software architecture 706, including implementation of the methods, components, and so forth described herein. The hardware layer 752 also includes memory and/or storage devices memory/storage 756, which also have executable instructions 704. The hardware layer 752 may also comprise other hardware 758. The software architecture 706 may be deployed in any one or more of the components shown in FIG. 1 or 2. The software architecture 706 can be utilized to detect and identify differences in data between two data sources using Merkle trees.

In the example architecture of FIG. 7, the software architecture 706 may be conceptualized as a stack of layers where each layer provides particular functionality. For example, the software architecture 706 may include layers such as an operating system 702, libraries 720, frameworks/middleware 718, applications 716, and a presentation layer 714. Operationally, the applications 716 and/or other components within the layers may invoke API calls 708 through the software stack and receive messages 712 in response to the API calls 708. The layers illustrated are representative in nature and not all software architectures have all layers. For example, some mobile or special purpose operating systems may not provide a frameworks/middleware 718, while others may provide such a layer. Other software architectures may include additional or different layers.

The operating system 702 may manage hardware resources and provide common services. The operating system 702 may include, for example, a kernel 722, services 724, and drivers 726. The kernel 722 may act as an abstraction layer between the hardware and the other software layers. For example, the kernel 722 may be responsible for memory management, processor management (e.g., scheduling), component management, networking, security settings, and so on. The services 724 may provide other common services for the other software layers. The drivers 726 are responsible for controlling or interfacing with the underlying hardware. For instance, the drivers 726 include display drivers, camera drivers, Bluetooth® drivers, flash memory drivers, serial communication drivers (e.g., Universal Serial Bus (USB) drivers), Wi-Fi® drivers, audio drivers, power management drivers, and so forth depending on the hardware configuration.

The libraries 720 provide a common infrastructure that is used by the applications 716 and/or other components and/or layers. The libraries 720 provide functionality that allows other software components to perform tasks in an easier fashion than to interface directly with the underlying operating system 702 functionality (e.g., kernel 722, services 724 and/or drivers 726). The libraries 720 may include system libraries 744 (e.g., C standard library) that may provide functions such as memory allocation functions, string manipulation functions, mathematical functions, and the like. In addition, the libraries 720 may include API libraries 746 such as media libraries (e.g., libraries to support presentation and manipulation of various media format such as MPREG4, H.264, MP3, AAC, AMR, JPG, PNG), graphics libraries (e.g., an OpenGL framework that may be used to render two-dimensional and three-dimensional in a graphic content on a display), database libraries (e.g., SQLite that may provide various relational database functions), web libraries (e.g., WebKit that may provide web browsing functionality), and the like. The libraries 720 may also include a wide variety of other libraries 748 to provide many other APIs to the applications 716 and other software components/devices.

The frameworks/middleware 718 (also sometimes referred to as middleware) provide a higher-level common infrastructure that may be used by the applications 716 and/or other software components/devices. For example, the frameworks/middleware 718 may provide various graphic user interface functions, high-level resource management, high-level location services, and so forth. The frameworks/middleware 718 may provide a broad spectrum of other APIs that may be utilized by the applications 716 and/or other software components/devices, some of which may be specific to a particular operating system 702 or platform.

The applications 716 include built-in applications 738 and/or third-party applications 740. Examples of representative built-in applications 738 may include, but are not limited to, a contacts application, a browser application, a book reader application, a location application, a media application, a messaging application, and/or a game application. Third-party applications 740 may include an application developed using the ANDROID™ or IOS™ software development kit (SDK) by an entity other than the vendor of the particular platform, and may be mobile software running on a mobile operating system such as IOS™, ANDROID™, WINDOWS® Phone, or other mobile operating systems. The third-party applications 740 may invoke the API calls 708 provided by the mobile operating system (such as operating system 702) to facilitate functionality described herein.

The applications 716 may use built-in operating system functions (e.g., kernel 722, services 724, and/or drivers 726), libraries 720, and frameworks/middleware 718 to create UIs to interact with users of the system. Alternatively, or additionally, in some systems, interactions with a user may occur through a presentation layer, such as presentation layer 714. In these systems, the application/component “logic” can be separated from the aspects of the application/component that interact with a user.

FIG. 8 is a block diagram illustrating components of a machine 800, according to some example embodiments, able to read instructions from a machine-readable medium (e.g., a machine-readable storage medium) and perform any one or more of the methodologies discussed herein. Specifically, FIG. 8 shows a diagrammatic representation of the machine 800 in the example form of a computer system, within which instructions 810 (e.g., software, a program, an application, an applet, an app, or other executable code) for causing the machine 800 to perform any one or more of the methodologies discussed herein may be executed. For example, the instructions 810 may be executed by the difference identification system 150 to detect and identify differences in data between two data sources using Merkle trees.

As such, the instructions 810 may be used to implement devices or components described herein. The instructions 810 transform the general, non-programmed machine 800 into a particular machine 800 programmed to carry out the described and illustrated functions in the manner described. In alternative embodiments, the machine 800 operates as a standalone device or may be coupled (e.g., networked) to other machines. In a networked deployment, the machine 800 may operate in the capacity of a server machine or a client machine in a server-client network environment, or as a peer machine in a peer-to-peer (or distributed) network environment. The machine 800 may comprise, but not be limited to a server computer, a client computer, a personal computer (PC), a tablet computer, a laptop computer, a netbook, a STB, a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 810, sequentially or otherwise, that specify actions to be taken by machine 800. Further, while only a single machine 800 is illustrated, the term “machine” shall also be taken to include a collection of machines that individually or jointly execute the instructions 810 to perform any one or more of the methodologies discussed herein.

The machine 800 may include processors 804, memory/storage 806, and I/O components 818, which may be configured to communicate with each other such as via a bus 802. In an example embodiment, the processors 804 (e.g., a central processing unit (CPU), a reduced instruction set computing (RISC) processor, a complex instruction set computing (CISC) processor, a graphics processing unit (GPU), a digital signal processor (DSP), an application-specific integrated circuit (ASIC), a radio-frequency integrated circuit (RFIC), another processor, or any suitable combination thereof) may include, for example, a processor 808 and a processor 812 that may execute the instructions 810. The term “processor” is intended to include multi-core processors 804 that may comprise two or more independent processors (sometimes referred to as “cores”) that may execute instructions contemporaneously. Although FIG. 8 shows multiple processors 804, the machine 800 may include a single processor with a single core, a single processor with multiple cores (e.g., a multi-core processor), multiple processors with a single core, multiple processors with multiple cores, or any combination thereof.

The memory/storage 806 may include a memory 814, such as a main memory, or other memory storage, local cache 152, and a storage unit 816, both accessible to the processors 804 such as via the bus 802. The storage unit 816 and memory 814 store the instructions 810 embodying any one or more of the methodologies or functions described herein. The instructions 810 may also reside, completely or partially, within the memory 814, within the storage unit 816, within at least one of the processors 804 (e.g., within the processor's cache memory), or any suitable combination thereof, during execution thereof by the machine 800. Accordingly, the memory 814, the storage unit 816, and the memory of processors 804 are examples of machine-readable media.

The I/O components 818 may include a wide variety of components to receive input, provide output, produce output, transmit information, exchange information, capture measurements. The specific I/O components 818 that are included in a particular machine 800 will depend on the type of machine. For example, portable machines such as mobile phones will likely include a touch input device or other such input mechanisms, while a headless server machine will likely not include such a touch input device. It will be appreciated that the I/O components 818 may include many other components that are not shown in FIG. 8. The I/O components 818 are grouped according to functionality merely for simplifying the following discussion and the grouping is in no way limiting. In various example embodiments, the I/O components 818 may include output components 826 and input components 828. The output components 826 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 828 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or other pointing instrument), tactile input components (e.g., a physical button, a touch screen that provides location and/or force of touches or touch gestures, or other tactile input components), audio input components (e.g., a microphone), and the like.

In further example embodiments, the I/O components 818 may include biometric components 839, motion components 834, environmental components 836, or position components 838 among a wide array of other components. For example, the biometric components 839 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram based identification), and the like. The motion components 834 may include acceleration sensor components (e.g., accelerometer), gravitation sensor components, rotation sensor components (e.g., gyroscope), and so forth. The environmental components 836 may include, for example, illumination sensor components (e.g., photometer), temperature sensor components (e.g., one or more thermometer that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 838 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 818 may include communication components 840 operable to couple the machine 800 to a network 837 or devices 829 via coupling 824 and coupling 822, respectively. For example, the communication components 840 may include a network interface component or other suitable device to interface with the network 837. In further examples, communication components 840 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 829 may be another machine or any of a wide variety of peripheral devices (e.g., a peripheral device coupled via a USB).

Moreover, the communication components 840 may detect identifiers or include components operable to detect identifiers. For example, the communication components 840 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 840, such as location via Internet Protocol (IP) geo-location, location via Wi-Fi® signal triangulation, location via detecting a NFC beacon signal that may indicate a particular location, and so forth.

### Glossary

“CARRIER SIGNAL” in this context refers to any intangible medium that is capable of storing, encoding, or carrying transitory or non-transitory instructions for execution by the machine, and includes digital or analog communications signals or other intangible medium to facilitate communication of such instructions. Instructions may be transmitted or received over the network using a transitory or non-transitory transmission medium via a network interface device and using any one of a number of well-known transfer protocols.

“CLIENT DEVICE” in this context refers to any machine that interfaces to a communications network to obtain resources from one or more server systems or other client devices. A client device may be, but is not limited to, a mobile phone, desktop computer, laptop, PDA, smart phone, tablet, ultra book, netbook, laptop, multi-processor system, microprocessor-based or programmable consumer electronics, game console, set-top box, or any other communication device that a user may use to access a network.

“COMMUNICATIONS NETWORK” in this context refers to one or more portions of a network that may be an ad hoc network, an intranet, an extranet, a virtual private network (VPN), a LAN, a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other type of cellular or wireless coupling. In this example, the coupling may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

“MACHINE-READABLE MEDIUM” in this context refers to a component, device, or other tangible media able to store instructions and data temporarily or permanently and may include, but is not limited to, random-access memory (RAM), read-only memory (ROM), buffer memory, flash memory, optical media, magnetic media, cache memory, other types of storage (e.g., Erasable Programmable Read-Only Memory (EEPROM)) and/or any suitable combination thereof. The term “machine-readable medium” should be taken to include a single medium or multiple media (e.g., a centralized or distributed database, or associated caches and servers) able to store instructions. The term “machine-readable medium” shall also be taken to include any medium, or combination of multiple media, that is capable of storing instructions (e.g., code) for execution by a machine, such that the instructions, when executed by one or more processors of the machine, cause the machine to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” excludes signals per se.

“COMPONENT” in this context refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example embodiments, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

A hardware component may also be implemented mechanically, electronically, or any suitable combination thereof. For example, a hardware component may include dedicated circuitry or logic that is permanently configured to perform certain operations. A hardware component may be a special-purpose processor, such as a Field-Programmable Gate Array (FPGA) or an ASIC. A hardware component may also include programmable logic or circuitry that is temporarily configured by software to perform certain operations. For example, a hardware component may include software executed by a general-purpose processor or other programmable processor. Once configured by such software, hardware components become specific machines (or specific components of a machine) uniquely tailored to perform the configured functions and are no longer general-purpose processors. It will be appreciated that the decision to implement a hardware component mechanically, in dedicated and permanently configured circuitry, or in temporarily configured circuitry (e.g., configured by software) may be driven by cost and time considerations. Accordingly, the phrase “hardware component”(or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware components are temporarily configured (e.g., programmed), each of the hardware components need not be configured or instantiated at any one instance in time. For example, where a hardware component comprises a general-purpose processor configured by software to become a special-purpose processor, the general-purpose processor may be configured as respectively different special-purpose processors (e.g., comprising different hardware components) at different times. Software accordingly configures a particular processor or processors, for example, to constitute a particular hardware component at one instance of time and to constitute a different hardware component at a different instance of time.

Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In embodiments in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output.

Hardware components may also initiate communications with input or output devices and can operate on a resource (e.g., a collection of information). The various operations of example methods described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” refers to a hardware component implemented using one or more processors. Similarly, the methods described herein may be at least partially processor-implemented, with a particular processor or processors being an example of hardware. For example, at least some of the operations of a method may be performed by one or more processors or processor-implemented components. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an API). The performance of certain of the operations may be distributed among the processors, not only residing within a single machine, but deployed across a number of machines. In some example embodiments, the processors or processor-implemented components may be located in a single geographic location (e.g., within a home environment, an office environment, or a server farm). In other example embodiments, the processors or processor-implemented components may be distributed across a number of geographic locations.

“PROCESSOR” in this context refers to any circuit or virtual circuit (a physical circuit emulated by logic executing on an actual processor) that manipulates data values according to control signals (e.g., “commands,” “op codes,” “machine code,” etc.) and which produces corresponding output signals that are applied to operate a machine. A processor may, for example, be a CPU, a RISC processor, a CISC processor, a GPU, a DSP, an ASIC, a RFIC, or any combination thereof. A processor may further be a multi-core processor having two or more independent processors (sometimes referred to as “cores”) that may execute instructions contemporaneously.

Changes and modifications may be made to the disclosed embodiments without departing from the scope of the present disclosure. These and other changes or modifications are intended to be included within the scope of the present disclosure, as expressed in the following claims.

The Abstract of the Disclosure is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separate embodiment.

What is claimed is:

1. A method comprising:
accessing, by one or more processors, a first set of data from a first source and a second set of data from a second source;
extracting, based on a key, a first subset of data from the first set of data and a second subset of data from the second set of data;
generating a first Merkle tree based on the first subset of data and a second Merkle tree based on the second subset of data;
comparing a first node from the first Merkle tree with a corresponding second node from the second Merkle tree; and
identifying one or more differences between the first subset of data and the second subset of data in response to determining that the first node fails to match the second node.

2. The method of claim 1, wherein the first source comprises at least one of a first database or a first streaming source comprising a first topic, and wherein the second source comprises at least one of a second database or a second streaming source comprising a second topic.

3. The method of claim 1, wherein the first set of data comprises a first set of JSON files and the second set of data comprises a second set of JSON files.

4. The method of claim 1, further comprising:
receiving the key;
accessing a configuration map that is associated with the key; and
extracting the first subset of data and the second subset of data according to the configuration map.

5. The method of claim 4, wherein the configuration map comprises a plurality of keys that define an organization of the extracted first subset of data and the extracted second subset of data.

6. The method of claim 1, wherein generating the first Merkle tree comprises:
generating a first hash value based on a first portion of the first subset of data;
generating a second hash value based on a second portion of the first subset of data;
generating a first root node of the first Merkle tree comprising a third hash value representing a combination of the first hash value and the second hash value; and
linking the first root node to the first portion of the first subset of data and the second portion of the first subset of data.

7. The method of claim 6, further comprising:
generating a fourth hash value based on a third portion of the first subset of data;
generating a fifth hash value based on a fourth portion of the first subset of data;
generating a second root node of the first Merkle tree comprising a sixth hash value representing a combination of the fourth hash value and the fifth hash value; and
linking the second root node to the third portion of the first subset of data and the fourth portion of the first subset of data.

8. The method of claim 7, further comprising:
generating a third root node of the first Merkle tree comprising a seventh hash value representing a combination of the third hash value and the sixth hash value; and
linking the third root node to the first root node and the second root node.

9. The method of claim 1, further comprising:
determining existence of the one or more differences between the first subset of data and the second subset of data in response to determining that the first node fails to match the second node; and
in response to determining the existence of the one or more differences, identifying the one or more differences between the first subset of data and the second subset of data by comparing additional nodes of the first Merkle tree with additional nodes of the second Merkle tree.

10. The method of claim 9, wherein comparing the additional nodes of the first Merkle tree with the additional nodes of the second Merkle tree comprises:
traversing a first subset of nodes of the first Merkle tree without traversing a second subset of nodes of the first Merkle tree; and
traversing a third subset of nodes of the second Merkle tree without traversing a fourth subset of nodes of the second Merkle tree.

11. The method of claim 10, further comprising:
determining that at least one node of the first subset of nodes is different from at least one node of the third subset of nodes;
determining that a root node the second subset of nodes is identical to a root node of the fourth subset of nodes; and
in response to determining that the root node the second subset of nodes is identical to the root node of the fourth subset of nodes, selecting paths along the first and second Merkle trees to traverse that comprise the first and third subsets of nodes.

12. The method of claim 1, further comprising:
identifying a first plurality of nodes of the first Merkle tree that branch off the first node along a first path;
identifying a second plurality of nodes of the first Merkle tree that branch off the first node along a second path;
identifying a third plurality of nodes of the second Merkle tree that branch off the second node along a third path; and
identifying a fourth plurality of nodes of the second Merkle tree that branch off the second node along a fourth path.

13. The method of claim 12, further comprising:
comparing a first hash value stored in a third node of the first plurality of nodes with a second hash value stored in a fourth node of the third plurality of nodes;
comparing a third hash value stored in a fifth node of the second plurality of nodes with a fourth hash value stored in a sixth node of the fourth plurality of nodes;
determining that the first hash value matches the second hash value and that the third hash value fails to match the fourth hash value; and
in response to determining that the first hash value matches the second hash value and that the third hash value fails to match the fourth hash value:
processing the second plurality of nodes along the second path and the fourth plurality of nodes along the fourth path; and
discontinuing processing the first plurality of nodes along the first path and the third plurality of nodes along the third path.

14. The method of claim 13, further comprising:
identifying a first pair of leaf nodes that are linked to an individual node of the second plurality of nodes that stores a different hash value from an individual node of the fourth plurality of nodes, the individual node of the fourth plurality of nodes being linked to a second pair of leaf nodes; and
comparing data values associated with the first pair of leaf nodes with data values associated with the second pair of leaf nodes to identifying the one or more differences between the first subset of data and the second subset of data.

15. The method of claim 1, wherein the first set of data comprises a first set of patient profile information and wherein the second set of data comprises a second set of patient profile information.

16. The method of claim 1, further comprising:
replicating the first set of data from the first source to the second source to generate the second set of data; and
identifying the one or more differences between the first subset of data and the second subset of data in response to replicating the first set of data from the first source to the second source.

17. The method of claim 1, further comprising:
receiving a plurality of events corresponding to different keys;
extracting different subsets of data from the first set of data for each of the plurality of events based on a respective one of the keys;
extracting different subsets of data from the second set of data for each of the plurality of events based on the respective one of the keys;
generating a first plurality of Merkle trees based on the different subsets of data from the first set of data for each of the plurality of events; and
generating a second plurality of Merkle trees based on the different subsets of data from the second set of data for each of the plurality of events.

18. The method of claim 17, further comprising:
detecting differences between the different subsets of data of the first and second sets of data by comparing the first plurality of Merkle trees with the second plurality of Merkle trees.

19. A system comprising:
one or more processors coupled to a memory comprising non-transitory computer instructions that, when executed by the one or more processors, perform operations comprising:
accessing a first set of data from a first source and a second set of data from a second source;
extracting, based on a key, a first subset of data from the first set of data and a second subset of data from the second set of data;
generating a first Merkle tree based on the first subset of data and a second Merkle tree based on the second subset of data;
comparing a first node from the first Merkle tree with a corresponding second node from the second Merkle tree; and
identifying one or more differences between the first subset of data and the second subset of data in response to determining that the first node fails to match the second node.

20. A non-transitory computer readable medium comprising non-transitory computer-readable instructions for performing operations comprising:
accessing a first set of data from a first source and a second set of data from a second source;
extracting, based on a key, a first subset of data from the first set of data and a second subset of data from the second set of data;
generating a first Merkle tree based on the first subset of data and a second Merkle tree based on the second subset of data;
comparing a first node from the first Merkle tree with a corresponding second node from the second Merkle tree; and
identifying one or more differences between the first subset of data and the second subset of data in response to determining that the first node fails to match the second node.
